(19) United States
(12) Patent Application Publication
(10) Pub. No.: US 2026/0158182 A1
(43) Pub. Date: Jun. 11, 2026

(54) AN AUTOMATIC HAND DISINFECTION DISPENSER, METHODS FOR USE OF THE AUTOMATIC HAND DISINFECTION DISPENSER AND A DISTRIBUTED HAND HYGIENE ASSISTANCE SYSTEM

(71) Applicant: Lagen Holding LTD, Gibraltar (GI)

(72) Inventor: Kjell Knoop, Lviv (UA)

(21) Appl. No.: 18/708,056

(22) PCT Filed: Nov. 8, 2022
(86) PCT No.: PCT/IB2022/060721

§ 371 Date: May 7, 2024

(30) Foreign Application Priority Data
Nov. 8, 2021 (NO) 20211341

(51) Int. Cl. A61L 2/18; A47K 5/12; A61L 2/24; A61L 103/07; H04W 4/80

(52) U.S. Cl. CPC A61L 2/18; A47K 5/1217; A61L 2/24; H04W 4/80; A61L 2103/07; A61L 2202/14

(57) ABSTRACT

The present invention relates to dispensing apparatuses and hygiene systems it provides an automatic hand washer 10, the automatic hand washer 10 is provided with means that detect persons approaching the automatic hand washer 10, present invention also provides a method for use of the automatic hand washer and a distributed hygiene system 140 which includes the automatic hand washer 10.

### TECHNICAL FIELD

The invention relates to the area of hand disinfection, and more precisely to an automatic hand disinfection dispenser and the use of the automatic hand disinfection dispenser, it is also disclosed a distributed hand hygiene assistance system including among others at least one automatic hand disinfection dispenser.

### BACKGROUND ART

Awareness of the importance of good hand hygiene in preventing the spread of infection has increased in recent years, not least in the wake of the Covid-19 pandemic. It is obviously important to be careful with hand hygiene in hospitals, but the need extends far beyond the walls of hospitals. Educational buildings, institutions, museums, passenger ships and many other places benefit from users being careful with hand hygiene.

In hospitals, it is not sufficient for staff to wash their hands regularly, it is necessary to disinfect hands when moving from patient to patient, and/or to use gloves.

Ensuring that one has performed a wash that is satisfactory in the context of infection is demanding, remembering to repeat the wash triggered by certain actions is even more demanding.

The World Health Organisation, an UN-organisation, has developed a scheme for hand hygiene at hospitals, old people's home, and similar institutions. The scheme is called the five moments. The five moments are:

TABLE 1

| | | | |
|---|---|---|---|
| 1. | Before | When | Clean your hands before touching a |
| | touching a | | patient when approaching him/her |
| | patient | Why | To protect the patient against harmful |
| | | | germs carried on your hands. |
| 2. | Before clean/ | When | Clean your hands immediately before |
| | aseptic | | performing a clean/aseptic procedure. |
| | procedure | Why | To protect the patient against |
| | | | harmful germs, including the patient's |
| | | | own, from entering his/her body. |
| 3. | After body | When | Clean your hands immediately |
| | fluid | | after an exposure risk to |
| | exposure | | body fluids (and after glove removal). |
| | risk | Why | To protect yourself and the |
| | | | health-care environment from |
| | | | harmful patient germs. |
| 4. | After | When | Clean your hands after touching |
| | touching | | a patient and her/his immediate |
| | a patient | | surroundings, when leaving the |
| | | | patient's side. |
| | | Why | To protect yourself and the |
| | | | health-care environment from |
| | | | harmful patient germs. |
| 5. | After | When | Clean your hands after touching |
| | touching | | any object or furniture in |
| | patient | | the patient's immediate |
| | surroundings | | surroundings, leaving-even if the |
| | | | patient has not been touched. |
| | | Why | To protect yourself and the |
| | | | health-care environment from |
| | | | harmful patient germs. |

Today's systems leave it to the individual to remember good hand hygiene and not least to follow the five moments where required.

The closest prior art gives examples of access control systems in combination with sanitizing stations. One example can be found in the patent application WO 2008/073562 A1, this document discloses a system for controlling access and recording of who has been allowed or denied access to controlled access areas it also shows a sanitization station which controls access to protected areas and records who have been allowed to enter and who has been denied entry to the area. US 2009/0219131 A1 discloses a system designed for use in hospitals for tracking hand washing and other medical care related tasks.

Current systems do not provide feedback to operators on compliance with hand hygiene rules, the systems are trust-based and there is little or no opportunity to reliably determine the effect of how the introduction of new hygiene regimes works.

It is an object of the present invention to provide solutions which solve the above-mentioned problems.

### DISCLOSURE OF INVENTION

It is one object of the invention to provide systems and methods that do not suffer from the problems indicated above. By introducing an automatic hand disinfection dispenser, the automatic is provided with means that detect persons approaching the automatic hand disinfection dispenser, it also comprises communication means and not at least two spray chambers for safe disinfection of hands. The automatic handwasher is intelligent in that it includes processing means and memory facilitating upgrading with new functions. The automatic hand disinfection dispenser may be remotely configured, and it may be provided with signalling means which can indicate mode and state of the hand disinfection dispenser in a visible way or in a visible and audible way, alternatively only in an audible way. The automatic hand disinfection dispenser facilitates messaging of the usage to a higher-level system, such as a computer system configured to operate one or more automatic hand disinfection dispensers.

As the automatic hand disinfection dispenser is provided with processors, communication means and memories it is well suited to be programmed to handle situation recognition based on logical evaluation of processes and alarming in case of recognition of hygienically critical situations.

Key features of a medical assistance system which includes one or more automatic handwashers can be:

The automatic hand disinfection dispenser, either as a stand-alone unit or as part in a system may find its use in:
cruise ships, ferries, trains, buses, taxis, planes
hospitals
farming, cattle, harvest hands
eateries including canteens
food industry
defense, barracks
emergency physicians
practice of physicians, dentists, veterinarians
nursing home care
infra structure hubs such as: port terminals, bus stations, train stations and airports
hotels
cinemas
churches
gyms, public swimming pools, fitness studios, sports clubs
private houses

In health institutions demands strict hand hygiene regimes, thus the automatic hand disinfection dispenser must include disinfection fluids for use in health institutions. In a particular embodiment the present invention provides an automatic hand disinfection dispenser. The present invention also includes methods to operate the automatic handwasher.

The automatic hand disinfection dispenser, due to its processing means, memory and communication means, may be one or more elements in a distributed hand hygiene assistance system in accordance with the present invention.

More particularly it is provided an automatic hand disinfection dispenser at least comprising:
a) a substantially closed housing that can be opened and closed, where the housing at least comprises:
   i. two spray chambers each with an open free end adapted to receive a hand and each with a closed end, the two spray chambers further comprise several spray nozzles;
   ii. a removable disinfectant container;
   iii. a power source providing electric power for operation of the automatic hand disinfection dispenser;
   iv. a control unit;
   v. “hand sensors”;
   vi. a fluidic system, and
   vii. a communication unit for wireless communication;
wherein the two hand sensors are configured to detect hands being inserted into the two spray chambers, If hand sensors are active “1” then the hand sensor is configured to transmit a trigger signal to the control unit of the automatic hand disinfection dispenser, the control unit is configured to initiate dispensing from the several spray nozzles of the two spray chambers in response to the received trigger signal, the fluidic system is configured to transport disinfectant from the removable disinfectant container to the several spray nozzles, and the fluidic system is also configured to generate pressure to spray the disinfectant onto a user's hands being inserted into the two spray chambers.

In one aspect of the invention the automatic hand disinfection dispenser comprises at least:
viii. a proximity sensor for detection of personnel in proximity of the automatic hand disinfection dispenser.

The communication unit can be one of:
a Wi-Fi unit in compliance with Wi-Fi standards;
cellular network in compliance with one of: GSM, GPRS, EDGE, WCDMA, 3G, 4G, 5G and H+;
Bluetooth;
Zigbee;
BLE;
RFID;
Z wave; and
LPWANS.

In some aspects of the invention the housing further comprises one or more of:
ix. at least one lightening source and user interface, where the lightening source and the user interface at least comprises:
   1. a group of lightening indicators visible for a user which is faced towards the front end of the automatic hand disinfection dispenser and not more than two meters away from the two spray chambers;
   2. a beacon light displaying status information of the automatic handwasher, and
   3. an internal interface, where the internal interface provides necessary controls and signals for maintenance purpose of the automatic hand disinfection dispenser;
x. at least one RFID reader;
xi. one or more audible sources;
xii. a metal detector integrated into each of the two spray chambers;
xiii. a disinfectant level sensor, detecting whether the disinfectant level falls below a certain threshold in the removable disinfectant container;
xiv. a disinfectant label scanner, the scanner scans a fixed area of the removable disinfectant container, the scanner is configured to scan 2D-codes, if a 2D code is present its data is saved in a memory of the automatic hand disinfection dispenser and wirelessly transmitted to an external access point;
xv. a removable dripping tray;
xvi. one or more memory circuits in communication with the control unit;
xvii. a housing open sensor, detects opening of the substantially closed housing;
xviii. a housing lock for unlocking and locking the substantially closed housing;
vii. a mode switch button, and
viii. an ambient light sensor.

The automatic hand disinfection dispenser can be configurable through a communication interface from an external access point. The communication interface can be wireless or wired such as ethernet. There are numerous wireless alternatives such as one off:
WLAN in accordance with Wi-Fi protocols,
Bluetooth,
Bluetooth mesh,
mobile telephone platform (such as but not limited to: GSM, GPRS, CDMA, EDGE, 3G, H+, 4G etc).

The automatic hand disinfection dispenser can be configurable from an external access point and configuration of the automatic hand disinfection dispenser may include one or more of the configuration parameters:
a) date and time;
b) user identification via RFID selectable to: on or off;
c) metal detection selectable to: on or off;
d) night mode, selectable start time for night mode and selectable stop time for night mode;
e) selectable audio volume;
f) all audible feedback is selectable to: on or off;
g) audible feedback user identification is selectable to: on or off;
h) negative audible feedback after metal detection is selectable to: on or off;
i) time delay from hand detection to start of spraying is selectable;
j) time delay when metal is detected for one or two hands from hand detection to start of spraying is selectable;
k) audible feedback after successful dispensing is selectable to one of: off and confirmation tone and audio file
l) time delay from end of dispensing to the restart of the hand disinfection process—if proximity sensor does not register that the user has left the automatic hand disinfection dispenser—is selectable;
m) audible feedback upon entering or exiting cleaning mode is selectable; and
n) number of cycles sprayed by the one removable disinfectant container until a level warning is activated is selectable.

The automatic hand disinfection dispenser can be configured to enter cleaning mode by activation of a push button on the housing.

According to one embodiment it is provided a method for operation of an automatic hand disinfection dispenser including two spray chambers including the steps of:
a) a first user approaches the automatic hand disinfection dispenser, and the automatic hand disinfection dispenser detects the approach utilising an embedded proximity sensor.
b) the automatic hand disinfection dispenser initiates an insert hand signal indicating that the first user shall insert hands into the two spray chambers.
c) in each spray chamber detecting by a hand sensor a hand being inserted into the spray chamber and the hand sensors triggers the automatic hand disinfection dispenser to enter spraying modes when hands are detected in one or both spray chambers;
d) the first user removes his disinfected hands and moves away when spraying process is finished.

Step b may further comprise the steps of: switching on a hand insertion LED light on the front of the automatic hand disinfection dispenser statically and starting to flash or run upwards a light strip.

Step c may further comprise the sub steps of: the automatic hand disinfection dispenser signals that it is in spraying mode by switching on a light strip statically, by turning off a hand insertion icon.

It is disclosed a method for automatic hand disinfection dispenser at least comprising the steps of:
a. providing a housing that can be opened and closed, at least comprising the steps of:
   i. providing two spray chambers (11a, 11b) each with an open free end adapted to receive a hand and each with a closed end, the two spray chambers (11a, 11b) further comprise several spray nozzles (121-129);
   ii. providing a removable disinfectant container (91);
   iii. providing a power source providing electric power for operation of the automatic hand disinfection dispenser;
   iv. providing a control unit;
   v. providing “hand sensors”;
   vi. providing a fluidic system, and
   vii. providing a communication unit for wireless communication with one or more automatic hand disinfection dispenser;
detecting hands being inserted into the two spray chambers utilising the two hand sensors, If hand sensors are active “1” then the hand sensor is configured to transmit a trigger signal to the control unit of the automatic hand disinfection dispenser, the control unit is configured to initiate dispensing from the several spray nozzles of the two spray chambers in response to the received trigger signal, where the fluidic system is configured to transport disinfectant from the removable disinfectant container to the several spray nozzles, and the fluidic system is also configured to generate pressure to spray the disinfectant onto a user's hands being inserted into the two spray chambers.

According to yet an embodiment of the invention it is provided a distributed hand hygiene assistance system at least comprising the following units:
a. at least one access point configured to receive and transmit data from one or more automatic hand disinfection dispensers, the access point further being configured to send commands to one or more automatic hand disinfection dispensers; and
b. at least one automatic hand disinfection dispenser where the automatic hand disinfection dispenser at least comprises:
   i. a substantially closed housing that can be opened and closed a where the substantially closed housing at least comprises:
      1. two spray chambers each with an open free end adapted to receive a hand and each with a closed end, the two spray chambers further comprise several spray nozzles;
      2. a disinfectant provider;
      3. a power source providing electric power for operation of the automatic hand disinfection dispenser, and
      4. a communication unit for wireless communication.

The distributed hand hygiene assistance system may in one aspect further comprise one or more of the following units:
c. at least one motion detector for the detection of persons, the motion detector is fixedly mounted,
d. at least one marker lights being visible and indicating whether a set hygiene regime has been followed or not;
e. at least one wireless communication device associated with the at least one motion detector and the at least one marker lights source;
where the automatic hand disinfection dispenser is at least configured to communicate with the motion detector.

The at least one motion detector can be one of: a video camera, an IR-sensor, PIR-sensor, laser motion sensor, microwave sensor and a lidar. A combination of multiple technologies in one can be used in an effort to improve reliability.

The distributed hand hygiene assistance system may further comprise:
a. a detection housing includes one motion detector, one processor, one wireless communication device, and at least one marker lights source,
b. the one wireless communication device is at least configured to communicate with the automatic hand disinfection dispenser,
c. the motion detector is connected with the processor for direct communication with the processor,
d. the processor is connected with the at least one marker lights, and the processor is configured to send commands to the at least one marker light, the commands are at least:
   if hygiene regime is violated send a trigger signal to the at least one marker lights to switch on an alerting light indicating the violation of the hygiene regime,
   if hygiene regime is followed send a trigger signal to the at least one marker lights to switch on a light indicating that the hygiene regime is followed.

The distributed hand hygiene assistance system may further at least comprise an RFID reader for registration of users associated with unique RFID chips.

Additional features and advantages of the present invention will be apparent from the accompanying claims.

### BRIEF DESCRIPTION OF DRAWINGS

Following is a brief description of the drawings in order to make the invention more readily understandable, the discussion that follows will refer to the accompanying drawings, in which

FIG. 1 shows an automatic hand disinfection dispenser seen in perspective,

FIG. 2 shows the automatic hand disinfection dispenser seen from front,

FIG. 3 shows on the right side of the figure the automatic hand disinfection dispenser seen from the left side and the automatic hand disinfection dispenser to the right of the figure is seen from the right side,

FIG. 4 shows cross section of the automatic hand disinfection dispenser seen in perspective,

FIG. 5 shows cross section of the automatic hand disinfection dispenser seen from the left side,

FIG. 6 shows the automatic hand disinfection dispenser activated with an RFID chip, the RFID chip being embedded in a smart card in this example,

FIG. 7 shows an example of activation of self-cleaning program for the automatic hand disinfection dispenser by push button on the automatic hand disinfection dispenser,

FIG. 8 shows an example of opening of the automatic hand disinfection dispenser, in this example with a key tool used on a lock,

FIG. 9 shows an example of an open automatic hand disinfection dispenser ready for maintenance,

FIG. 10 shows the open automatic hand disinfection dispenser with interior service and maintenance button being activated,

FIG. 11 shows removal of drip tray for cleaning or service in the open automatic hand disinfection dispenser,

FIG. 12 shows spray chamber seen with 8 nozzles for wetting hands with disinfectants,

FIG. 13 shows a medical assistance system comprising several automatic hand disinfection dispensers and an access point,

FIG. 14 shows an example of a room with the automatic hand disinfection dispenser, an RFID antenna and a sensor mounting bracket including 2 range finder sensors,

FIG. 15 shows a patient room, including sensors, automatic hand disinfection dispensers, light markers and personnel,

FIG. 16 shows a patient room including Wi-Fi transmitters, Wi-Fi receivers, light markers source, proximity sensor, one bed and one automatic hand disinfection dispenser associated with the bed,

FIG. 17 shows a patient in a bed and two nurses one which has followed a hand cleansing regime according to the present invention and one which has not, the latter being indicated with an alarm light,

FIG. 18 shows a patient sitting on a bed, where a light marker from the ceiling shows an alarm light, indicating that the patient has not followed a hand disinfection regime,

FIG. 19 overview of a distributed medical assistance system (left), and

FIG. 20 shows a patient room, including sensors, wash basins and automatic hand disinfection dispenser and light markers.

### DETAILED DESCRIPTION OF THE INVENTION

In the following it is firstly disclosed general embodiments in accordance with the present invention, thereafter particular exemplary embodiments will be described. Where possible reference will be made to the accompanying drawings and where possible using reference numerals in the drawings. It shall be noted however that the drawings are exemplary embodiments only and other features and embodiments may well be within the scope of the invention as described.

The applicant has realised that the problem with hand hygiene is more complex than only providing a good hand dispenser, the hand dispenser needs to communicate with the users and thereby inform users whether disinfection has been successful. Further, in systems with several hand dispensers it will be an advantage to include communication means in the hand dispenser, where the communication system can forward status to a central system.

The present invention provides an automatic hand disinfection dispenser for stand-alone operation and for operation in complex systems comprising multiple handwashers, surveillance system, one or more access points and signalling systems where the complex system includes communication means for communication between units of the complex system.

The strict hand hygiene regime at health institutions demands the use of a disinfection apparatus, whilst in less demanding environment an automatic hand disinfection dispenser with soap may be sufficient. In the following description the wording automatic hand disinfection dispenser is used as a generic term for the automatic hand disinfection dispenser. The automatic hand disinfection dispenser may communicate with signals to users, indicating that an approaching user needs to disinfect his or her hands, the automatic handwasher will give feedback by signalling if the disinfection has been successful or not. The automatic hand disinfection dispenser may indicate with signalling its status, i.e., if it needs to be refilled if cleaning or other maintenance is necessary. The signalling may be provided with lighting signals, with audible signals or with a combination of lighting signals and audible signals.

In a more complex system including several automatic handwashers the signalling to users may be provided with lighting signals, with audible signals or with a combination of lighting signals and audible signals. The more complex systems are tailored to comply with the five moments of WHO.

In the following an automatic hand disinfection dispenser both for stand-alone operation and for operation in systems comprising several automatic handwashers according to the present invention will be described. More complex systems will be described separately.

### The Automatic Hand Disinfection Dispenser

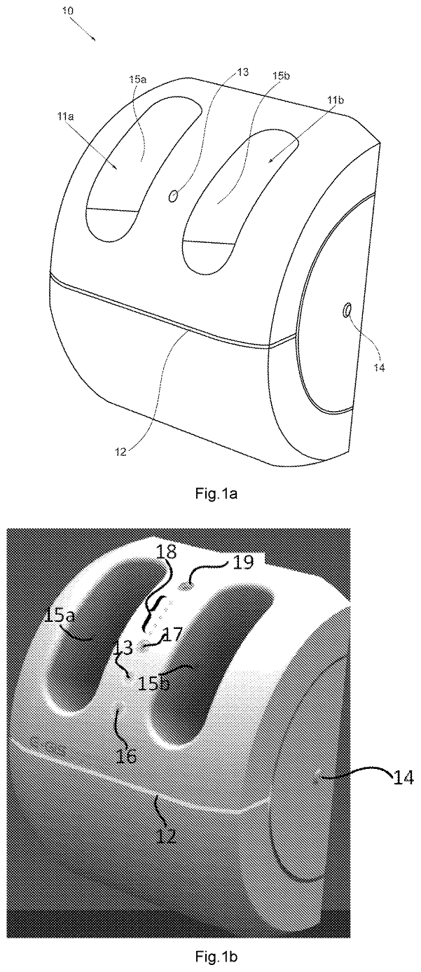

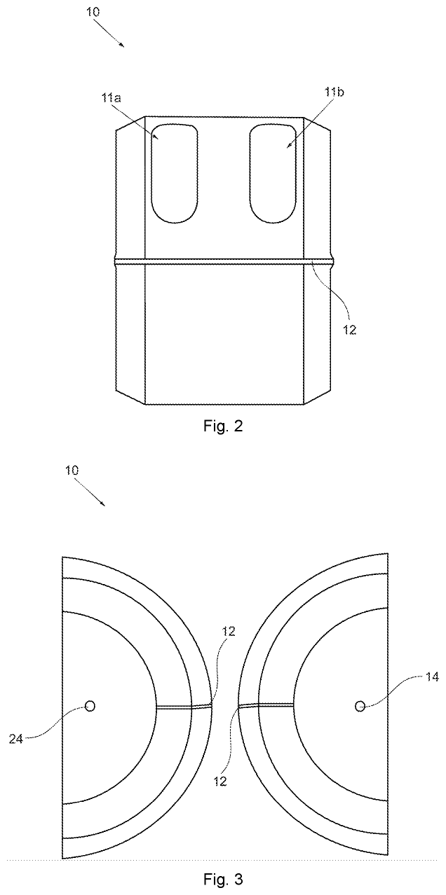

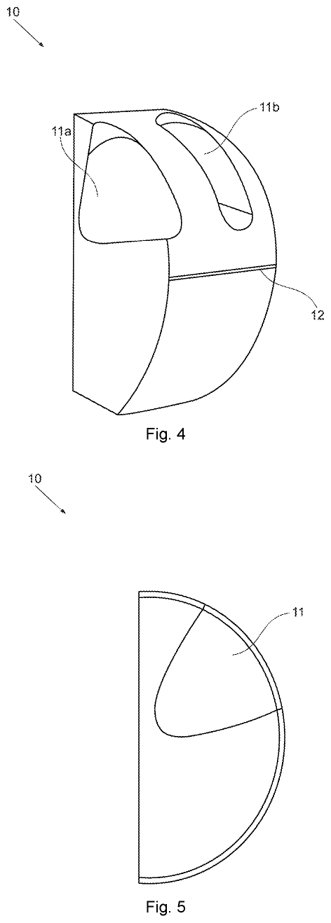

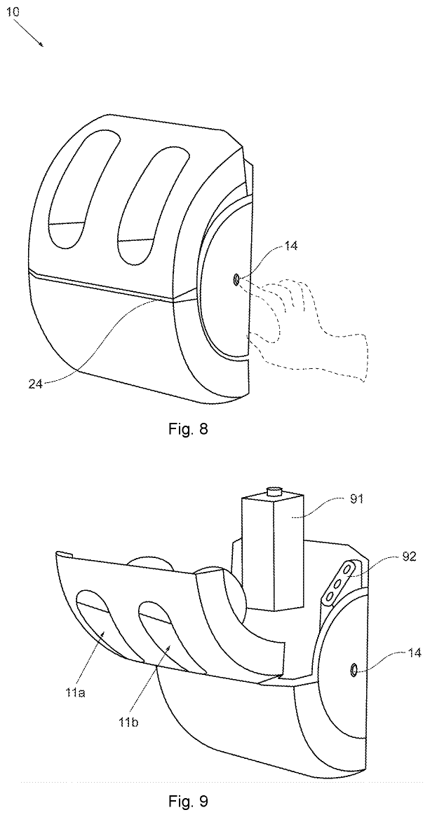

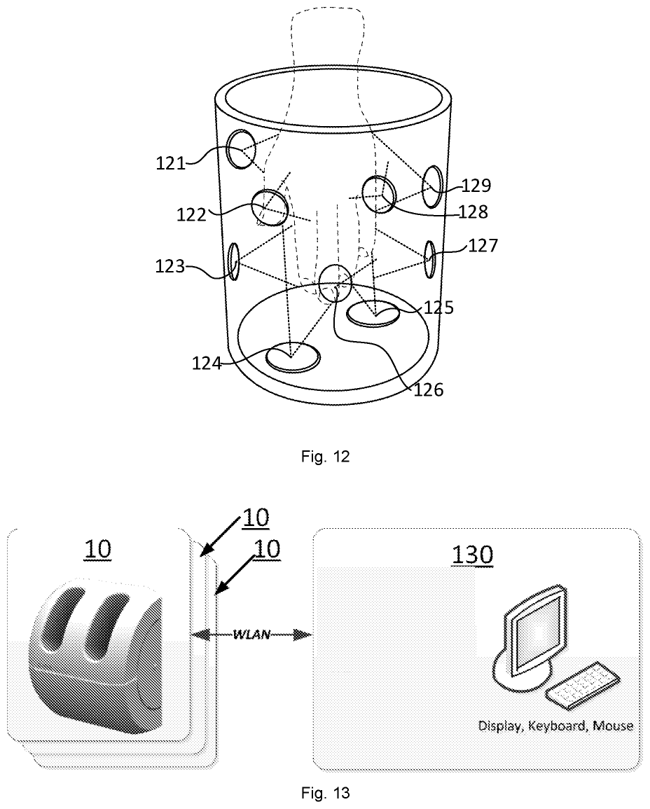

A first embodiment of an automatic hand disinfection dispenser will now be described with reference to FIGS. 1-12.

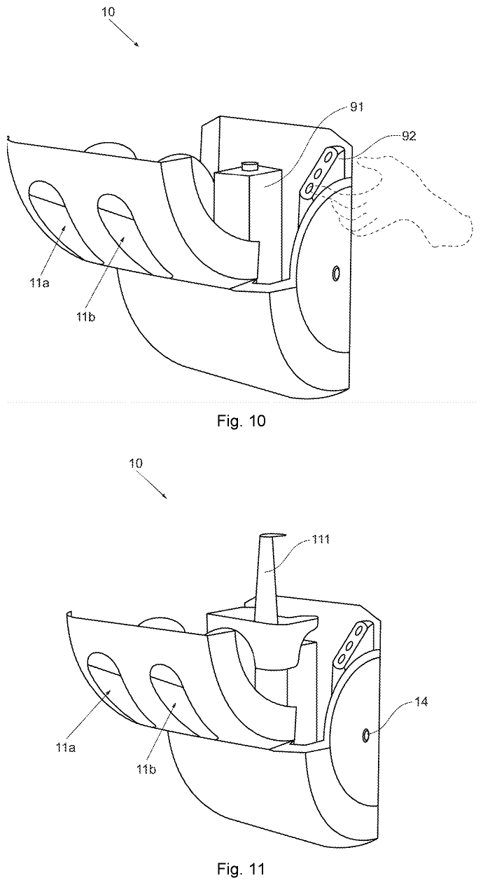

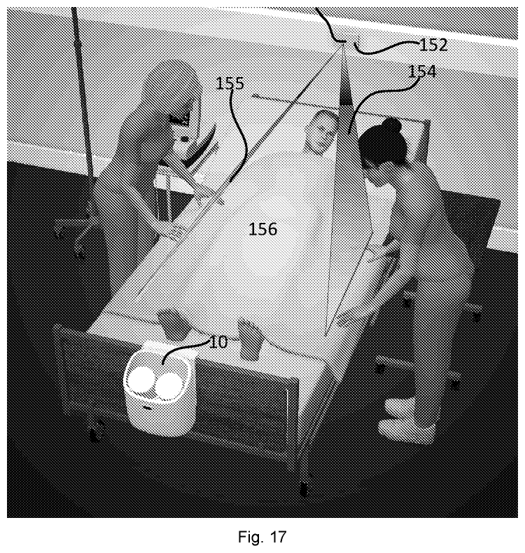

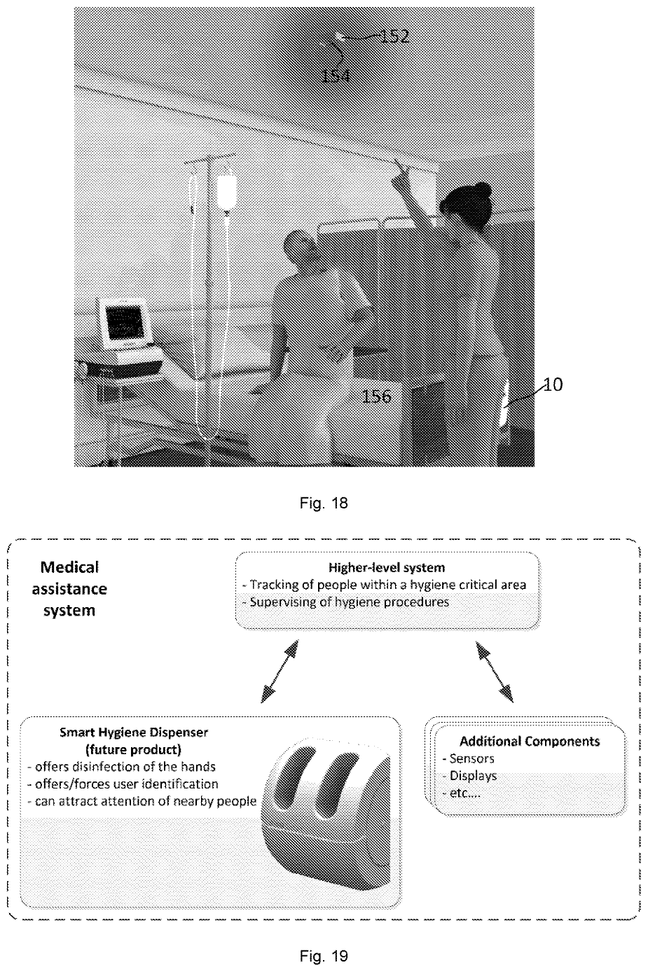

FIG. 1 shows an example of an automatic handwasher 10 another automatic hand disinfection dispenser 10 with a different exterior design is shown in FIG. 17. The exterior design of the housing is only limited by the functionality of the automatic hand disinfection dispenser 10. There are some functionalities of the automatic hand disinfection dispenser 10 that according to the present invention must be present. The automatic hand disinfection dispenser 10 must include a housing where service staff can have access to the interior of the automatic hand disinfection dispenser. FIGS. 8-11 shows an example of a substantially closed housing of an automatic handwasher 10, where the substantially closed housing can be opened and closed. In the example shown the housing is opened with a key adapted for a key lock 14. The housing of the automatic handwasher may comprise a housing open sensor which detects opening of the substantially closed housing. A beacon light 12 on the automatic handwasher may light up to indicate that the automatic handwasher is not closed and/or locked, in addition to or as an alternative an audible signal or a stored voice may indicate that the automatic handwasher 10 is not closed.

For operation of the automatic handwasher 10 with all its features it is necessary to equip the automatic handwasher with a power source. The power source may be one or more of: an internal transformer, an external transformer and a chargeable battery pack. The embodiment with internal transformer is connectable to mains. For security reasons the automatic handwasher 10 may be provided with chargeable back-up batteries which ensures safe operation in black outs.

Each automatic handwasher 10 is provided with a proximity sensor, if the sensor is enabled it will be activated if one or more persons approaches one automatic hand disinfection dispenser 10. If a control unit of the automatic hand disinfection dispenser 10 has been configured to indicate persons approaching the automatic handwasher 10, then the control unit will trigger alert signals, the alert signals can be one or more of:
Switch on a beacon light 12 on the automatic hand disinfection dispenser 10 so that the automatic handwasher 10 attracts attention,
a ready signal 17 indicating that the automatic handwasher is ready for disinfection can be switched on,
a group of lightening indicators visible for a user which is faced towards the front end of the automatic hand disinfection dispenser and not more than two meters away from the two spray chambers. The group of lighting can be arranged as an array of lights 18, where the array 18 lights up sequentially to advice a proximate person to insert his hands into the spray chambers. The array of lights can be switched on simultaneously with the ready signal and or the beacon light signal in bullet point one above.
An audible alert, or a spoken recommendation and/or command.
A visible alert on a display for displaying a recommendation and or command.

The automatic hand disinfection dispenser 10 is provided with “hand sensors”, the object of the “hand sensors” are to detect insertion of hands in the spray chambers 11, 11a, 11b and to provide the detection of hands by signalling to the control unit. The control unit is in communication with one or more memory circuits.

The control unit in response will initialise dispensing fluid within a specified time interval. Signal lights and audible signals may indicate that hands have been detected in the spray chambers 11. 11a, 11b, and lights or audible signals may indicate ongoing spraying and finally indicate that spraying is finished. Each spray chamber 11, 11a, 11b is associated with its own hand sensor. A time delay from hand detection to start of spraying is selectable by configuration from the external access point 130 and/or from an internal interface 92. The time delay from end of dispensing to the restart of the hand disinfection process—if proximity sensor does not register that the user has left the automatic hand disinfection dispenser—is selectable by configuration from an external access point 130 and/or from an internal interface 92.

The automatic hand disinfection dispenser 10 is configurable from the external access point 130, additionally the hand disinfection dispenser is provided with an internal interface 92, the internal interface can provide the necessary controls and signals for maintenance purpose. To communicate with the external access point 130 the automatic hand disinfection dispenser needs communication means. In one aspect of the invention the communication means is provided as Wi-Fi communication units, in this aspect of the invention the automatic hand disinfection dispenser 10 is provided with a Wi-Fi-transceiver in compliance with common Wi-Fi standards. The access point 130 can be a computer with necessary peripherals such as keyboards and a screen and a Wi-Fi transceiver. Software for control and configuration of one or more automatic hand disinfection dispensers 10 must be accessible from the access point 130. In complex systems the access point 130 will receive and store operational data from several automatic hand disinfection dispensers 10 and from other units in a complex distributed hand hygiene assistance system 140. The acquired data can be used for maintenance purposes, i.e. refill disinfectant, clean automatic hand disinfection dispensers 10 or order disinfectant, the acquired data can be invaluable when looking at the extent to which users meet the rules for hand hygiene, in the longer term one will in some systems be able to look at direct effects between the spread of infection and the enforcement of hygiene regimes where automatic hand disinfection dispensers 10 are used together with other components in a distributed hand hygiene assistance system 140.

The automatic hand disinfection dispenser 10 comprises two spray chambers 11. In the embodiment shown in FIGS. 1-12 it is shown a left spray chamber 11a and a right spray chamber 11b. The walls of the spray chambers are provided with holes (FIG. 1b) 15a, 15b adapted to receive nozzles 121-129. The FIGS. 1-11 do not show holes for all nozzles, it shall be understood that each nozzle is associated with a hole in the spray chamber walls. It is important that the nozzles/holes are distributed so that disinfectant reach all parts of the hands, including under the nails and between the fingers.

The spray chambers are associated with one or two metal detectors, one detector per chamber or one shared detector, and the automatic hand disinfection dispenser may be configured to activate the one or two metal detectors. This feature can be deactivated by software. If the metal detectors are activated, then a control unit of the automatic hand disinfection dispenser will be triggered to carry out one or more of the following steps:
The automatic handwasher will notify deviations from specific routines such as deviations from the five moments. Indications of deviations can be indicated with light signals 19 on the automatic hand disinfection dispenser 10 and/or with audible signals. Additionally, the automatic hand disinfection dispenser 10 may transmit the deviation to an access point 130 if the automatic hand disinfection dispenser 10 is part of a distributed hand hygiene assistance system 140.

A disinfectant supply is necessary for operation of the automatic handwasher 10, hence the automatic handwasher 10 is provided with one or more disinfectant containers, in one embodiment the automatic handwasher 10 is provided with one removable disinfectant container/bottle 91. The one or more disinfectant containers are in fluidic communication with an internal fluidic system, where the internal fluidic system includes several nozzles 121-129. The fluidic system is configured to generate pressure to spray the disinfectant onto a user's hands being inserted into the chambers 11. For the convenience of maintenance persons, the automatic handwasher can be provided with a disinfectant level sensor, detecting whether the disinfectant level falls below a certain threshold in the one or more disinfectant containers. Furthermore, to ensure that the correct disinfectant fluid is used in the automatic handwasher 10 the automatic handwasher 10 may be provided with a disinfectant label scanner, the scanner scans a fixed area of the one or more disinfectant containers, the scanner can be configured to scan 2D-codes, if a 2D code is present its data can be saved in a memory of the automatic hand disinfection dispenser 10 and may be wirelessly transmitted to an external access point 130. To prevent disinfectant discharge from coming out of the housing or meeting unwanted parts in the housing of the automatic handwasher 10, the automatic handwasher 10 may be equipped with a removable dripping tray 111.

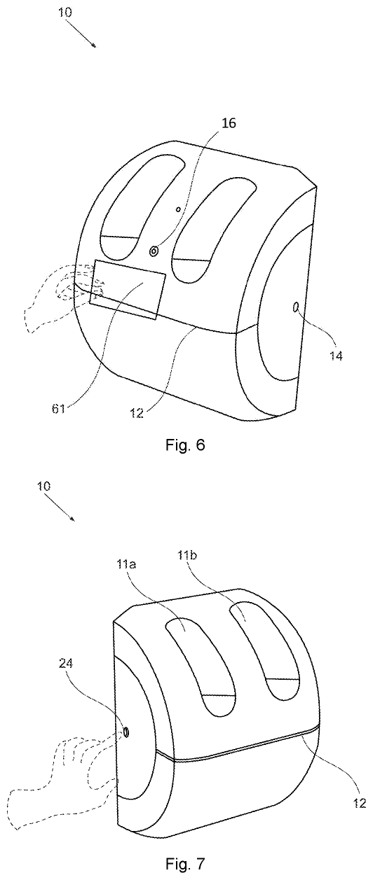

The automatic hand disinfection dispenser may be provided with an RFID reader. The RFID reader may be enabled from an access point 130 and/or from an internal interface unit 92 of the automatic hand disinfection dispenser 10. RFID readers may in a convenient way log users that activates the RFID reader by a RFID chip 61, see FIG. 6. In the distributed hand hygiene assistance system 140 it is a matter of software implementation which functionality a system shall have with respect to registration of RFID chips and storing data related to use of RFID chips in the access point 130.

The automatic hand disinfection dispenser 10 may be provided with a mode switch button 24. Activation of the mode switch button 24 can change the state of the automatic handwasher. The chosen state may be indicated by light signals on the automatic handwasher 10, for example by the beacon light 12—by switching colour and/or by starting to pulsate. Other signals such as digital displays and/or audible signals may also be activated. Among others the automatic hand disinfection dispenser 10 can be configured to enter cleaning mode by activation of the push button 24 on the housing. In a concrete example pushing the mode switch button for 5 second will set the automatic handwasher in a maintenance mode and the beacon lights 12 starts blinking indicating that the automatic hand disinfection dispenser 10 is in maintenance mode and not operational and surface cleaning can be performed now.

To adapt the automatic hand disinfection dispenser to patient rooms etc. it can be provided with an ambient light sensor, where output signals from the ambient light sensor can be assessed by the control unit and the control unit may adjust audible signalling and not at least the strength of the lightning. The output from the ambient light sensor may be transmitted to the access point 130 in a system which includes an access point.

### Lighting and User Interface of the Automatic Hand Disinfection Dispenser

For a visual feedback to the user, three basic elements are integrated into the automatic hand disinfection dispenser 10. One of these elements is a group of indicators 13, 16, 17, 18, 19, which is faced towards the user. These indicators give feedback during the normal use of the automatic hand disinfection dispenser. The second basic element is called beacon light 12 and its purpose is to display general information while the automatic hand disinfection dispenser 10 is in use or is not in use. The third element is located inside the automatic hand disinfection dispenser 10 and it displays information to the maintenance personnel or cleaning personnel, while the housing of the automatic hand disinfection dispenser 10 is opened. The third element is referred to as internal interface 92.

To compensate the brightness of the indicators at different ambient light brightness, an ambient light sensor can be integrated into the automatic hand disinfection dispenser 10. The output of this sensor is coupled with the brightness of the indicators.

### Characteristics of the Access Point

The purpose of the access point 130 is to provide a basic means to communicate within a distributed hand hygiene assistance system.

The access point 130 can be based on a single-board computer. For its operation, further peripheral devices are required, namely a monitor, a keyboard and, if necessary, a mouse, see FIG. 13. The communication between the automatic hand disinfection dispenser 10 and the access point 130 can be based on anyone of Wireless LAN, Bluetooth and mobile telephone platform such as GSM, GPRS, CDMA EDGE, 3G etc. During operation, the automatic hand disinfection dispenser 10 can send messages to the access point 130, which can be observed by an operator of the access point 130 via the monitor. The operator can also send messages to the automatic hand disinfection dispenser 10. With these messages, the configuration of the automatic hand disinfection dispenser 10 can be changed, for example, to deactivate a metal detection feature. Additionally, these messages can also contain commands, which initiate actions of the automatic hand disinfection dispenser 10, like instructing the automatic hand disinfection dispenser 10 to use or not use an inserted disinfectant cartridge or setting the time for example.

The access point 130 may allow one or more of the following features:
Receiving of status information from the automatic hand disinfection dispenser 10.
Sending of commands to the automatic hand disinfection dispenser 10.
Changing of the configuration of the automatic hand disinfection dispenser. The configuration and date/time can be saved non-volatile in the automatic hand disinfection dispenser and the automatic hand disinfection dispensers may be pre-configured before shipping.

One or more of the parameters in the bullet points below can be configured on the automatic hand disinfection dispenser 10 by the access point 130. For example, in the first bullet point the date and time will be set for the automatic hand disinfection dispenser 10. The remaining bullet point give the configuring person options to switch on or off features of the automatic hand disinfection dispenser 10. Obviously, the features are examples and the use of a control unit combined with internal memories in the automatic hand disinfection dispenser 10 opens for even more advanced features such as displaying messages and using voice messages.
i. Date and time
ii. User identification via RFID: on/off
iii. Metal detection: on/off
iv. Night mode: start and stop time/off
v. Audio volume
vi. All audible feedback: on/off
vii. Audible feedback user identification: on/off
viii. Negative audible feedback after metal detection: on/off
ix. Time delay from hand detection to start of spraying (regular, no metal detected)
x. Time delay from hand detection to start of spraying (metal detected)
xi. Audible feedback after successful dispensing: off/confirmation tone/audio file
xii. Time delay from end of dispensing to the restart of the hand disinfection process (if proximity sensor does not register that the user has left the automatic hand disinfection dispenser 10)
xiii. cleaning mode—button pressing time to enter or exit
xiv. cleaning mode—audible feedback upon entering or exiting
xv. cleaning mode—auto exit: off/time delay
xvi. Spraying cycle counter-number of cycles until level icon lights up red.

### States and Modes of the Automatic Hand Disinfection Dispenser

The automatic hand disinfection dispenser 10 includes components that facilitates set up of modes and states of the automatic hand disinfection dispenser 10. The automatic hand disinfection dispenser at least includes a processor, memory in communication with the processor and communication means. The communication means can be WLAN and/or ethernet.

### Installation, Initial Setup

Attach the dispenser to a convenient location so that it is easily accessible to users. If the dispenser is not of a rechargeable type, it must be placed so that it can access the mains. In configurations that include access point 130 the (WLAN) configuration to connect with the access point 130 is permanently set in the automatic hand disinfection dispenser 10. If necessary, the device settings of the automatic hand disinfection dispenser 10 can be changed via the access point 130.

The automatic hand disinfection dispenser has a user interface, in the figures the user interface is the internal user interface 92, however, the design and location of the user interface is a matter of design of the automatic hand disinfection dispenser 10.

To allow for audible feedback during the use of the automatic hand disinfection dispenser, an audio device can be integrated into the automatic hand disinfection dispenser 10. Additionally, the playback of audio files can be initiated by the access point 130, and automatically played after each use of the automatic hand disinfection dispenser 10. This is only regarding to the automatic hand disinfection dispenser 10 and its original function.

In an embodiment disclosed in FIGS. 1-13 the connection status of the automatic hand disinfection dispenser 10 can be displayed via access point 130 status icon, (on the internal interface 92 of the automatic hand disinfection dispenser 10):
Connection to the access point 130 exists: static green.
Connection to the access point 130 does not exist: static red

After closing the housing, the automatic hand disinfection dispenser 10 will enter an idle state.

### Idle State

In the idle state is the automatic hand disinfection dispenser 10 is ready for use. An automatic handwasher 10 with beacon light will show the idle state with a particular “idle state” signal. The idle state is described with reference to FIGS. 1-13. The signalling is a matter of design options and other types of signalling can be envisioned for idle state. In the embodiment as disclosed in FIGS. 1-13 the beacon light 12 indicates (by slow pulsing: breathing) the operational readiness of the automatic hand disinfection dispenser 10. Depending on the configuration, it may be desirable that no or very low illumination is active in the idle state for example in night mode.

Upon receiving message of the type shown in table 8 below, an audio file is played by the automatic hand disinfection dispenser 10. A message of the type shown in table 9 below initiates a flashing of the beacon light 12.

Important display/operating elements:
Beacon Light (blue breathing, brightness depending on ambient light); audio output and/or optical signal (optional)
Preconditions: Initial setup.

### Night Mode

The automatic hand disinfection dispenser 10 may be configured to enter night mode at certain times in the evening. In night mode, the automatic hand disinfection dispenser operates as described for other modes/states. This means that the automatic hand disinfection dispenser 10 can be in idle state and night mode simultaneously. The difference between normal mode and night mode is that all confirmation and warning tones are switched off. In the idle state, the brightness of all light elements is reduced further, and any flashing (status) lights, if present, are replaced by dim continuous lights.

The night mode is intended for locations where constant bright lights, flashing lights and audible feedback are undesirable and might be annoying. If enabled via the configuration, the night mode is entered automatically between a period of the day (configurable via regular starting time and stopping time of the period of the day).

Preconditions: Night mode has been configured and activated with appropriate time settings.

Hand Disinfection with Active RFID User Identification

The automatic hand disinfection dispenser 10 may be provided with an RFID reader and if provided with such a reader activation of the reader is a matter of configuration. In some systems 140 separate RFID readers 141 may be installed for example at the entrance of a room. RFID readers are particularly helpful where the users of the automatic hand disinfection dispenser are a defined group of people.

It should be possible to identify a user via RFID tag. This option may be turned off.

When a user approaches the automatic hand disinfection dispenser 10 the automatic hand disinfection dispenser 10 will detect the approach due to an embedded proximity sensor. In addition to the hand icon 17 plus running light array 18, the user-ID LED 16 also starts to flash yellow when the user ID configuration is active.

The user is prompted to identify himself by the user-ID LED 16. The user-ID LED 16 signals the positive identification back to the user via a colour change to green (continuous illumination) and (optional, configurable) a positive signal tone (audio output).

If the user starts the disinfection process by inserting their hands without identification having taken place, the user-ID LED 16 (when hands are detected) lights up red and a single “negative” signal tone (audio output, optional, configurable) sound.

The disinfection process can be performed in any case.

Termination of the process is indicated by the hand insertion and user identification LEDs 16 and the light strip turning off and (optional, configurable) a positive signal tone (audio output).

After the spraying of the disinfectant is finished a message of the type shown in table 2 is sent from the automatic hand disinfection dispenser 10 to the access point 130 and the message is additionally saved to the local log of the automatic hand disinfection dispenser 10.

The user removes his hands and moves away. After (configurable) 1 sec, the idle state follows, provided that no other person is detected in front of the device. Otherwise, the process described above starts again.

Disinfection with Metal Detection (with or without User Identification)

The use of the automatic hand disinfection dispenser 10 with metal detection differs from normal use without metal detection in that as soon as the automatic hand disinfection dispenser 10 detects a metal object such as jewellery watches etc, a metal detection LED 19 starts to flash red and (optional, configurable) a single “negative” signal tone sound. The disinfection process starts as usual despite the detection of metal, but only after a (configurable) delay time. The metal detection LED 19 lights up statically after the delay time.

After the spraying of the disinfectant is finished, message type as shown in table 2 is sent to the access point 130 and is additionally saved to the local log in the memory of the automatic hand disinfection dispenser 10.

When the user removes his hands and if still being in front of the device, the insertion LED 17 and the light array 18 again prompt the insertion of the hands. If the user moves away, then the automatic hand disinfection dispenser 10 switches to idle state after a configurable time.

### Disinfectant Almost Empty

The automatic hand disinfection dispenser is provided with one or more disinfectant level sensors, depending on the number of disinfectant containers 91 that the automatic hand disinfection dispenser is designed for. The level sensor(s) will detect whether the disinfectant level falls below a certain threshold. With this information provided to the maintenance personnel, a better organization and planning of the regular refilling of the disinfectant should be achieved.

A contact-less, capacitive measuring principle is intended for the implementation of the level sensor. With this measuring principle, no additional components have to be integrated in the disinfect containers or the fluidic system and thus, their complexity is not increased by the level sensor.

In another variant the threshold value for the amount can be mechanically determined by the shape of the disinfectant container(s) and the position of the level sensor(s).

When the level falls below the threshold value, the disinfectant level LED lights 13 up yellow statically.

The message type in table two contains information about the disinfectant level and is transmitted after every use. Thus, no additional message is needed for “disinfectant almost empty”-state.

### Disinfectant Empty

The automatic hand disinfection dispenser 10 continuously counts the spraying cycles performed. The associated count value is reset as soon as the “Refill disinfectant” mode or the reprocessing mode has been performed.

As soon as the counter value has exceeded a configurable value, the fill level LED 13 lights up permanently red. The yellow glow of the LED due to the use case “Disinfectant almost empty” is overwritten by the permanent red glow.

The message type in table two contains information about the disinfectant level and is transmitted after every use. Thus, no additional message is needed for “disinfectant empty”-state.

Communication from the Automatic Hand Disinfection Dispenser to the Access Point

The automatic hand disinfection dispenser 10 is configurable from the external access point 130 as indicated in the previous section. This applies to distributed hand hygiene assistance system 140 which among others includes access point 130, stand-alone automatic hand disinfection dispensers 10 can be preconfigured according to specification provided by the buyer/owner.

In the distributed hand hygiene assistance system 140 the automatic hand disinfection dispenser 10 will communicate with the access point using particular protocols, examples of communication protocols are given below:

### Message Type Usage of Dispenser

After the spraying of the disinfectant is finished the messages in table 2 below is transmitted from the automatic hand disinfection dispenser to the access point 130 and is additionally saved to a local log in the local memories of the automatic hand disinfection dispenser 10.

TABLE 2

| No. of | | |
|---|---|---|
| data | | |
| field | description | value(s) |
| 1 | ID of automatic hand | |
| | disinfection dispenser 10 | |
| 2 | Message Date | YYYY-MM-DD_hh:mm |
| 3 | metal detected | one out of: |
| | | no |
| | | yes |
| | | inactive |
| 4 | User ID | one out of: |
| | | ID read via RFID |
| | | no RFID tag found |
| | | inactive |
| 5 | dispensed dosages since | |
| | last refill of disinfectant | |
| 7 | disinfectant level | one out of: |
| | sensor output | level low |
| | | level not low |
| 8 | dosage counter | one out of: |
| | threshold reached | threshold not reached |
| | | threshold reached |

### Message Type Cleaning Mode

The automatic hand disinfection dispenser 10 must be cleaned periodically dependent on frequency of use, the environment of the location and the relevant hand hygiene regime. In one particular embodiment cleaning and/or wiping of an automatic hand disinfection dispenser 10 is carried out by cleaning personnel by pressing the button 24 on the left side of the housing for (configurable) 5 seconds, the automatic hand disinfection dispenser 10 is set to cleaning mode. This can be indicated by a static yellow glow of the beacon light 12 and/or as a short audio signal (optional, configurable).

The unit remains in this mode without disinfection or other user interaction, whilst the cleaning personnel wipes the surface (incl. the spray chambers 11) of the automatic hand disinfection dispenser 10.

The cleaning mode is ended by pressing the mode switch button 24 again for (configurable, see above) 5 seconds. (Audio signal, optional, configurable).

If the device is in cleaning mode for longer than e.g. 3 min (configurable timeout) (and the device has not been opened), the cleaning mode (configurable) is ended automatically.

After the automatic hand disinfection dispenser has entered cleaning mode, which may be activated using the mode switch button 24 on the automatic hand disinfection dispenser 10, the automatic handwasher 10 sends the message in table 3 to the access point 130 and the message is also saved to a local log in the local memories of the automatic hand disinfection dispenser 10.

TABLE 3

| No. of | | |
|---|---|---|
| data | | |
| field | description | value(s) |
| 1 | ID of automatic hand | |
| | disinfection dispenser 10 | |
| 2 | Message Date | YYYY-MM-DD_hh:mm |
| 3 | cleaning mode | one out of: |
| | | activated |
| | | deactivated |

### Message Housing Open or Closed

Messages relating to opening and closing of the housing are relevant for maintenance purposes such as refilling of disinfectant and reprocessing etc. Below follows schemes for different maintenance operations.

### Refill Disinfectant

In one particular embodiment the following scheme is followed when maintenance personnel open the housing of the automatic hand disinfection dispenser 10 with a key for reprocessing. The automatic hand disinfection dispenser 10 detects the opening and deactivates normal interactions (similar to the cleaning mode).

After opening the housing, a message of the type “table 4” is sent from the automatic hand disinfection dispenser 10 to the access point 130 and is additionally saved to the local log of the automatic hand disinfection dispenser 10.

Cleaning personnel removes one or more of the disinfectant containers 91 and empties a drip tray 111. The drip tray 111 and one or more new containers 91 of disinfectant are inserted.

After a switch is actuated by insertion of the one or more containers 91, the label reader reads the label of the one or more containers, the reading may be carried out at intervals of one second (maximum x times, e. g. x=3).

If a connection between the automatic hand disinfection dispenser 10 to the access point 130 is established, message type table 5 is sent to the access point and is additionally saved to the local log of the dispenser directly after the successful or unsuccessful scanning attempt. The access point 130 responds by sending a “message receipt” if data field 5 of table 4 is sent from the automatic hand disinfection dispenser 10 to the access point 130, then the access point 130 is responding by sending the message indicated in table 7. The received message is also saved to the local log of the automatic hand disinfection dispenser 10.

If the connection to the access point is not established, the dispenser decides, based on the best before date, which is stored in the scanned label data, if the one or more new containers 91 is allowed for use and is able to set a label icon accordingly. In this case, message type “table 5” is saved to the local log of the automatic hand disinfection dispenser 10.

Indication is given via the label reader LED which is turned on to indicate one or more valid containers 91 (such as green) if the one or more containers have been identified as invalid/prohibited/unidentifiable the label reader LED will be turned on to indicate this (such as with red light).

Optionally, a user may push a flush fluidic system button on the internal interface 92 to be able to remove old hand disinfectant or other cleaning agent from the tubes of the fluidic system, if necessary, the beacon light will indicate this by a light associated with the mode, for example by starting to flash yellow.

The housing is closed and locked. When the housing is closed, the status LEDs inside the housing are switched off, in the FIGS. 9 and 10 on the internal interface 92. Immediately after closing, the initiated flushing process is performed, if the corresponding button on the internal interface 92 was pressed.

After closing the housing, message type “table 4” is sent to the access point and is additionally saved to the local log of the dispenser. Following this the automatic hand disinfection dispenser 10 automatically switches to idle state.

### Reprocessing:

In one particular embodiment the following scheme is followed when maintenance personnel open the housing of the automatic hand disinfection dispenser 10 with a key for reprocessing. The automatic hand disinfection dispenser 10 detects the opening and deactivates normal interactions (similar to the cleaning mode).

After opening the housing, the message in table 4 is sent to the access point 130 and is additionally saved to the local log in the local memory of the automatic handwasher 10. After every opening or closing during a use case, this message type (Table 4) is sent to the access point 130 and is additionally saved to the local log of the dispenser.

The maintenance personnel may remove one or more of the disinfectant containers 91. If one or more of the disinfectant containers 91 is removed, then the maintenance personnel will insert a container with a special reprocessing agent. The Label of this container carries a special identification, which can be read by the label scanner of the automatic hand disinfection dispenser 10.

If the connection to the access point 130 is established, then the message type of table 4 is sent to the access point 130 and is additionally saved to the local log in the local memory of the automatic handwasher 10 directly after the successful or unsuccessful scanning attempt.

If the connection to the access point 130 is not established, the automatic hand disinfection dispenser decides, based on the disinfection type, which is stored in the scanned label data, if the one or more new containers 91 contains the reprocessing agent and is able to set the label icon accordingly. In this case a message of the type in table 5 is saved to the local log of in the local memory of the automatic hand disinfection dispenser 10.

If the agent for reprocessing is recognized by the corresponding label (label scanner) a label indicator such as label LED lights up in a particular colour such as blue. The automatic hand disinfection dispenser does not allow a disinfection process as long as this agent remains inserted.

Maintenance personnel initiate a fluidic system flush process by pressing a “Flush fluidic system” button on the internal interface 92, the beacon light 12 indicates the flush fluid mode, in a particular configuration the beacon light begins to flash yellow. The flushing process with the reprocessing agent is started as soon as the housing is closed.

After flushing with the reprocessing agent, the automatic hand disinfection dispenser 10 remains in cleaning mode and the beacon light 12 continues indicating flush fluid mode (such as yellow static) until the one or more containers with the reprocessing agent has been removed, the hand disinfectant to be used has been inserted and the housing has been closed.

After the application time of the reprocessing agent has elapsed, the one or more containers with the reprocessing agent is replaced by one or more containers 91 with hand disinfectant. The label reader reads the label of the one or more container 91, the reading is indicated with a label reader LED.

If the connection to the access point is established, message type shown in table 5 is sent to the access point 130 and is additionally saved to the local log of the automatic hand disinfection dispenser 10 directly after the successful or unsuccessful scanning attempt. The access point 130 responds by sending a “message receipt” if data field 5 of table 4 is sent from the automatic hand disinfection dispenser 10 to the access point 130, then the access point 130 is responding by sending the message indicated in table 7. When the automatic hand disinfection dispenser 10 receives the message from the access point it will be able to set a label icon accordingly. The received message is also saved to the local log of the automatic hand disinfection dispenser 10.

If the connection to the access point is not established, the automatic hand disinfection dispenser 10 decides, based on the best before date, which is stored in the scanned label data, if the one or more new containers 91 is allowed for use and is able to set the label icon accordingly. In this case, a message type as shown in table 5 is saved to the local log of the automatic hand disinfection dispenser 10.

TABLE 4

| No. of | | |
|---|---|---|
| data | | |
| field | description | value(s) |
| 1 | ID of automatic hand disinfection | |
| | dispenser 10 | |
| 2 | Message Date | YYYY-MM-DD_hh:mm |
| 3 | housing state | one out of: |
| | | opened |
| | | closed |
| 4 | flushing of the fluidic | one out of: |
| | system started | flushing started |
| | | flushing not started |
| 5 | one or more containers | one out of: |
| | 91 inserted | container inserted |
| | | no container inserted |

### Message Label Information

TABLE 5

| No. of | | |
|---|---|---|
| data | | |
| field | description | value(s) |
| 1 | ID of Automatic hand disinfection | |
| | dispenser 10 | |
| 2 | Message Date | YYYY-MM-DD_hh:mm |
| 3 | scanned data | one out of: |
| | | scanned label data |
| | | no label was scanned |

### Message Error Detected

If the automatic hand disinfection dispenser 10 enters error mode (out of order) all functions such as manual disinfection, flushing, metal detection, etc. are deactivated. All indication lights (icon LEDs) 13, 16, 17, 18, 19 outside are off.

### Out of Order (Error State) May Mean:

### No Disinfectant Container Inserted:

After opening or closing the housing, message type shown in table 4 is sent to the access point 130 and is additionally being saved to the local log of the automatic hand disinfection dispenser 10. This message type contains information about whether one or more containers 91 are inserted or not.

### Error:

As soon as the automatic hand disinfection dispenser 10 detects a hardware or software error itself, this can be indicated by the beacon light 12 glowing permanently red. After detection of the error a message of the type shown in table 6 is sent to the access point 130 and is additionally saved to the local log of the dispenser.

TABLE 6

| No. of | | |
|---|---|---|
| data | | |
| field | description | value(s) |
| 1 | ID of Automatic hand disinfection | |
| | dispenser 10 | |
| 2 | Message Date | YYYY-MM-DD_hh:mm |
| 3 | Type of Error | |

Communication from the Access Point to the Automatic Hand Disinfection Dispenser

The access point 130 sends receipt messages as responses to the automatic hand disinfection dispenser 10 sending state and/or mode messages to the access point 130. Also, the access point may send configuration settings to one or more automatic hand disinfection dispensers 10 in a distributed hand hygiene assistance system 140.

### Message Label Answer

The message in table 7 is typically sent in response to a message from table 5 sent by the automatic hand disinfection dispenser 10. Typically, the message in table 7 is sent during refill of disinfectant action and during use of reprocessing agent on one or more automatic hand disinfection dispensers 10.

TABLE 7

| No. of | | |
|---|---|---|
| data | | |
| field | description | value(s) |
| 1 | ID of Automatic hand disinfection | |
| | dispenser 10 | |
| 2 | label answer | one out of: |
| | | allowed |
| | | not allowed |
| | | reprocessing agent |

### Message Play Audio File

Upon receiving this message, the addressed (ID of automatic hand disinfection dispenser) automatic hand disinfection dispenser 10 plays one of the stored audio files to draw attention to itself acoustically, if the relevant automatic hand disinfection dispenser 10 is in idle state.

TABLE 8

| No. of | | |
|---|---|---|
| data | | |
| field | description | value(s) |
| 1 | ID of Automatic hand disinfection | |
| | dispenser 10 | |
| 2 | audio file to be played | name of file |

### Message Flash Beacon Light

Upon receiving this message, the addressed (ID of automatic hand disinfection dispenser) automatic hand disinfection dispenser 10 flashes the beacon light 5 times to draw attention to itself visually, if the addressed automatic hand disinfection dispenser 10 is in idle state.

TABLE 9

| No. of | | |
|---|---|---|
| data | | |
| field | description | value(s) |
| 1 | ID of Automatic hand disinfection | |
| | dispenser 10 | |

### Message Configuration

This message type is used to change the configuration of the automatic hand disinfection dispenser 10.

TABLE 10

| No. of | | |
|---|---|---|
| data | | |
| field | description | value(s) |
| 1 | ID of Automatic hand disinfection | |
| | dispenser 10 | |

### Distributed Hand Hygiene Assistance System

This section describes a distributed hand hygiene assistance system, which allows for improved compliance of the personnel with hygiene rules. FIG. 19 shows a rough sketch of this system. A higher-level system communicates with several components, like sensors, display elements and one or more automatic hand disinfection dispensers 10. A potential function of the whole distributed hand hygiene assistance system could be the tracking of persons inside a hospital room, while simultaneously monitoring the compliance to hygiene rules for every person. For example, as soon as a person attempts to approach or touch a patient without disinfected hands, the system can detect this critical violation of hygiene rules. Subsequently, corrective measures could be initiated, like warning the personnel about this critical situation. In the FIG. 17, this is done by projecting a red line 154 onto the patient's bed.

A minimum distributed hand hygiene assistance system consists of two basic device types, namely the access point 130 and one or more automatic hand disinfection dispensers 10 which are depicted in FIG. 13. The most important device is the automatic hand disinfection dispenser 10, which is shown on the left. The right side shows a device, which is named access point 130. One or more automatic hand disinfection dispenser 10 devices can connect to the access point 130, please refer to sections above.

FIGS. 13-15 shows overviews of different layouts of a distributed hand hygiene assistance system 140, the systems disclosed in these figures includes sensors installed in walls or roofs in rooms, moreover it includes lighting sources having the capacity to emit light in different colours and thereby indicate by the choice of lighting colour a state such as to indicate if persons in the room has followed a particular hygiene regime.

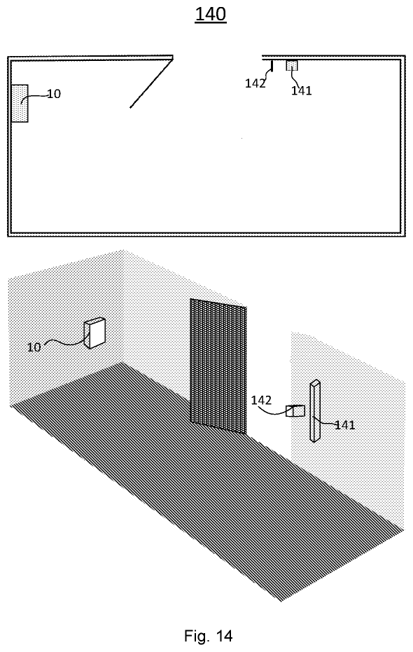

FIG. 14 shows a simple setup in one room-such as a doctor's room-including an RFID antenna and reader 141, 142 close to the entrance, thus the RFID reader will be able to read RFID chips entering the room. On one wall an automatic hand disinfection dispenser 10 is mounted. The system only discloses the units in the room, hence the access point 130 with which the automatic hand disinfection dispenser communicates is not disclosed.

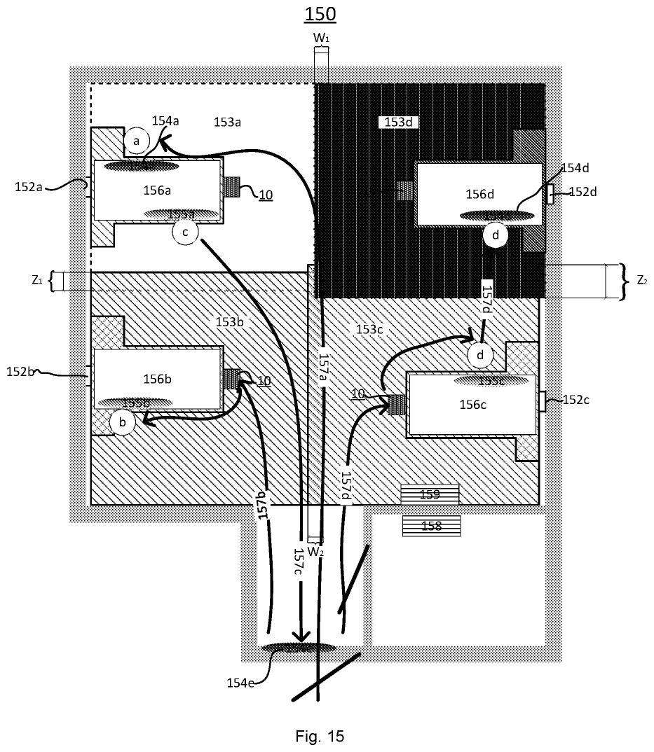

FIG. 15 shows a room 150 with four beds 156a, 156b, 156c, 156d, and four automatic hand disinfection dispensers 10 mounted to the respective beds, lighting sources are indicated 154a-154e, 155a-155c and a first 158 and second 159 sink.

The lightning sources may be embedded in the roof, the walls or alternatively the lighting sources and four sensors 152a-152d can be one single unit embedded in the walls or the roof.

The room includes four sensors 152a-152d shown located on the walls behind the beds 156a-156d. These sensors can be any type of motion sensors, their object is to detect persons entering their respective detecting areas. The sensor 152a covers an area denoted 153a, which is the area around the first bed 156a, the sensor 152b is associated with the area 153b, the sensor 152c is associated with the area 153c and sensor 152d is associated with area 153d. The areas 153a-153d is overlapping with neighbouring zones. There is an overlap between 153a and 153d indicated as W₁, between 153a and 153b indicated as Z₁, between 153b and 153c indicated as W₂ and finally between 153c and 153d indicated as Z₂. All zones, W₁, Z₁, W₂ and Z₂ are shown with an overlap. The zones may vary, it is however important that the substantially full area of the room is covered, and that it is possible to associate persons entering the room with areas thereby making it possible to track individuals within the room.

The motion sensors can be lidars or cameras or other motion detectors.

The automatic hand disinfection dispenser 10 in a distributed hand hygiene assistance system 140 includes communication means such as WLAN receivers and transmitters (ref. communication protocols from table 2-10 above), hence, to be operative and to communicate with an access point the room 150 must facilitate communication between the automatic hand disinfection dispensers 10 and the access point.

In one variant the motions sensors 152a, the lightning sources 155a/154 and communication means are included in one single unit, the same applies for zone 153b, 153c and 153d. The communication unit can be any type of wired or wireless communication unit such as but not limited to WLAN, Bluetooth, Bluetooth mesh, Ethernet, mobile telephone platform (such as but not limited to: GSM, GPRS, CDMA, EDGE, 3G, H+, 4G etc.).

The combination of automatic hand disinfection dispensers 10, sensors and communication means facilitate tracking of people and also if the tracked person follows a requested hygiene regime. FIG. 15 shows arrows, each arrow 157a, 157b, 157c, 157d indicates the path of one single person. A first person a enters the room through an entrance door and follows the path 147a directly to a patient in bed 156a. The person a has not washed his hands, i.e., has not been registered at the automatic hand disinfection dispenser on the first bed 156a before going to the patient in the first bed 156a. As a consequence of the violation of hygiene regimes the lighting source 154a is turned on as the person a approaches the first bed 156a. The lighting source 154a is a lighting source indicating that the hygiene routines have been violated. In one version the light can be red, however other colours can be chosen and also lighting patterns can be used to indicate violence of hand hygiene routines.

The person c follows a path 147c directly from the bed 156 and out of the room 150, thus exiting a room and touching a door directly from patient contact, this is not in compliance with the rules of the example in FIG. 15, thus a lighting 154e is turned on at the exit of the room thereby indicating that the exiting person have to disinfect his hands.

A person b enters the room and follows the path 157b directly to an automatic hand disinfection dispenser 10 proximate to or on the bed 156b, the person b disinfects his hands and continues to the patient in the second bed 156b, a light 155b is switched on to signal that the person b has followed the hygiene routines.

A person d enters the room 150 follows the path 157d directly to an automatic hand disinfection dispenser on or close to the bed 156c where the person d disinfects his hands and continues directly to a patient in the third bed 156c a light 155c is switched on to signal that the person d has followed the hygiene routines. The person d continues along the path 157d towards the fourth bed 156d without disinfecting his hands thus on arrival at the fourth bed a lighting source 154d is turned on as the person a approaches the fourth bed 156a. The lighting source 154d is a lighting source indicating that the hygiene routines have been violated.

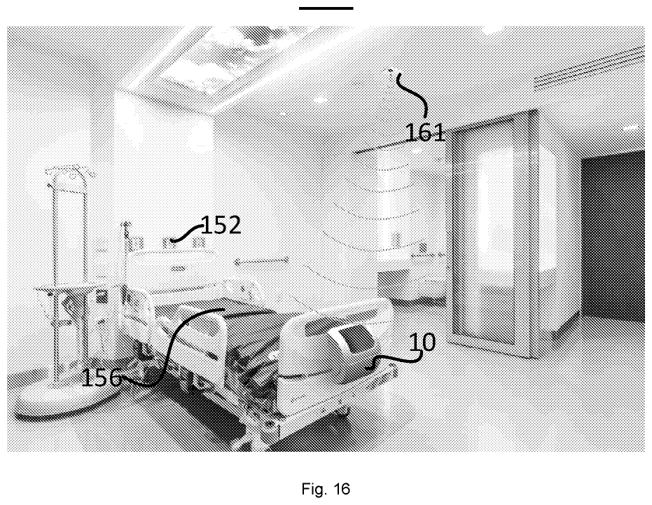

FIG. 16 shows a patient room with a distributed hand hygiene assistance system 140. A bed is indicated with reference number 156. A sensor in communication with processing means and a wireless communication unit 161 configured to communicate with the processing means is shown in the ceiling. In the wall behind the bed a lighting source 152 is situated the lighting source must be in operatively communication with the sensor and the wireless communication unit 161 and directly or indirectly in contact with the automatic hand disinfection dispenser 10. The lighting source includes communication means.

FIG. 17 shows the situation from the zone 153a in FIG. 15. The person on the left side of the bed (with light hair) has followed routines for hand hygiene consequently a “green” light 155 flashes her side of the bed. The person on the left-hand side has not followed the same routine and a “red” light up on the right-hand side of the bed. The figure shows a light source 170 on the wall behind the patient. The light source is designed to emit lights in different colours to both sides of the bed 156.

In this particular embodiment the light source does not include a sensor.

### Monitoring a Patient Room Using Smart Sensors and AI

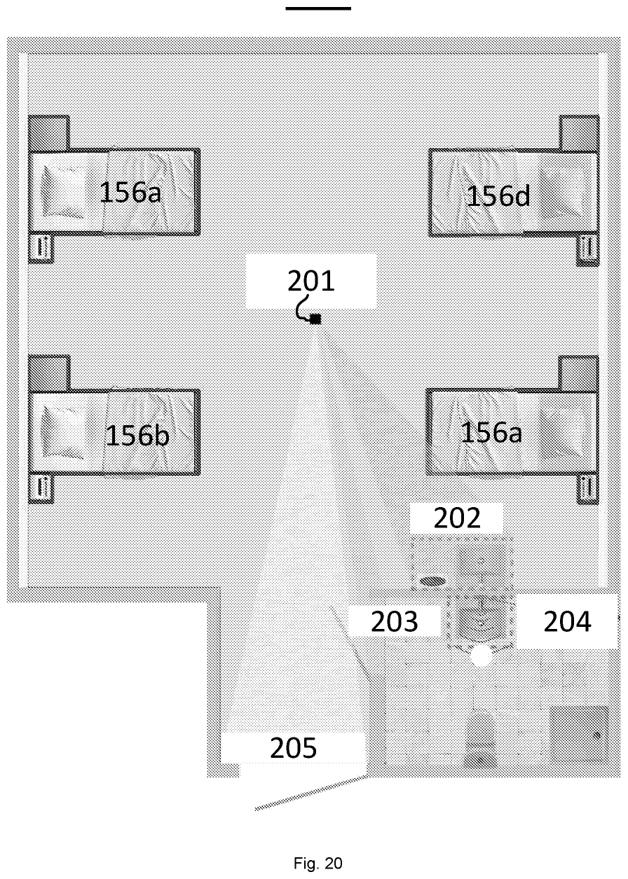

FIG. 20 shows a patient room with a sensor 201 combined with a processor and communication means mounted in the ceiling. The figure shows two washing stations-one in the patient room and one in a bathroom.

Both disinfection with soap and water as well as the use of the automatic disinfection dispenser can be approved ways to perform hand hygiene. To find out if a sink (soap and water) is used, a distance meter 204 can be mounted on the wall just above the sink. The distance meter will detect if someone is standing in front of the sink, and how long that person is standing there.

The zone 205 denoted the yellow zone monitors the entrance door area. Another yellow zone 203 monitors the entrance to the bathroom. A pink zone 202 monitors a washing station in the room, the washing station includes the automatic hand disinfection dispenser and a sink.

The sensor 201 can be trained to learn and monitor special situations and patterns, this is made possible by practical training where people perform desired and unwanted actions, the response from the sensor is analysed by the processor 201 either internally or in a central computer programmed for the task so the monitoring of the room improves over time.

Figure List of definitions, unless otherwise explicitly expressed in the description the following definitions applies:

Maintenance personnel:
Installs the automatic hand disinfection dispenser 10,
performs initial setup, is responsible for maintenance
and reprocessing at specified intervals

| | |
|---|---|
| Tasks | Installation, setup, maintenance, preparation, configuration. |

Cleaning personnel:
Regular/daily surface cleaning or wiping of the automatic hand
disinfection dispenser 10,

| | |
|---|---|
| Tasks | Cleaning/wiping, refilling |

User
Can be a guest, staff or a doctor, for example.
Uses the automatic hand disinfection dispenser 10 for hand disinfection,

| | |
|---|---|
| Tasks | Hand disinfection |
| LED | Light Emitting Diode |

List of reference numbers referring to the appended drawings and further explanations of aspects related to technical features of the present invention.

| | |
|---|---|
| Hand | The hand sensors according to the present invention are |
| sensor | sensors that are able to detect hands being inserted into spray |
| | chambers (11a, 11b). Different types of proximity sensors can |
| | be used such as non-contact sensors that |
| | detects the presence of an object when the object |
| | enters the sensor's field. The field can be created |
| | as sound field, light field, infrared radiation (IR), or |
| | electromagnetic fields. Many types of proximity sensors can |
| | thus be used to detect hands being inserted in the spray |
| | chambers may be utilized by the sensor to detect a target. |
| Housing | The actual cabinet or shell that makes up the housing that |
| | includes all the elements of the hand dispenser (10). |
| Prox- | The automatic hand disinfection dispenser |
| imity | can be provided with proximity sensors to |
| sensors | detect personnel approaching the automatic |
| | hand disinfection dispenser. The proximity sensor can be of |
| | different types, see row above with reference to hand sensors |
| | which gives examples of proximity sensors. |
| 10 | Automatic hand disinfection dispenser |
| 11 | Spray chambers of automatic hand disinfection dispenser 10 |
| 11a | Left spray chamber of automatic hand |
| | disinfection dispenser 10, seen from the front of |
| | the automatic hand disinfection dispenser |
| 11b | Right spray chamber of automatic hand disinfection dispenser |
| | 10, seen from the front of the automatic hand disinfection |
| 12 | Beacon light, the beacon light shows status of the automatic |
| | hand disinfection dispenser. Beacon light can have different |
| | colours and it can pulsate/blink. |
| 13 | Fill level LED, indicates status fill level of |
| | disinfectant remaining in the dispenser. In one |
| | example constant yellow indicates low |
| | level, whilst blinking red starts after a count of further uses. |
| 14 | Housing lock. Unlocking to open the automatic hand |
| | disinfection dispenser for refill of maintenance purposes. |
| 15a | Nozzle in left spray chamber for wetting hands |
| | with disinfectants |
| 15b | Nozzle in right spray chamber for wetting hands with |
| | disinfectants |
| 16 | Identification light, in one example blue blinking signal ready |
| | to read RFID card, static green indicates a positive reading. |
| 17 | Identification light “insert led”, in one particular example this |
| | light starts to glow when a person is detected, and it signals |
| | readiness for disinfection. The sequential lights |
| | 18 starts to run simultaneously. When a user |
| | inserts his/her hands into the spray |
| | chambers up to the right position the spraying process will be |
| | initiated. The LED will turn green to indicate successful |
| | disinfection. |
| 18 | Sequential lights, in one example the line of lights starts to |
| | glow together with the “insert led” 17. |
| 19 | In one example this light is referred to as jewellery led which |
| | starts pulsing red if metal is detected on the inserted hands. |
| 24 | In one example the automatic hand disinfection dispenser is |
| | provided with a button on the left side. The button, when |
| | activated, can set the automatic hand disinfection |
| | dispenser in maintenance mode. The beacon light |
| | 12 starts blinking indicating that the automatic |
| | hand disinfection dispenser is in maintenance |
| 91 | A removable disinfectant container. |
| 92 | Internal interface, the internal interface can provide the |
| | necessary controls and signals for maintenance purpose. |
| 111 | Removable dripping tray. |
| 121-129 | Nozzles. Examples of nozzles in one spray chamber for |
| | complete wetting of hands with disinfectants. |
| 130 | Access point. WLAN access point for configuration of |
| | automatic hand disinfection dispenser, observation of user |
| | data. The access point is not mandatory in smaller systems. |
| 140 | A distributed hand hygiene assistance system, the system may |
| | include several rooms each with: automatic hand disinfection |
| | dispensers and/or RFID reader and one or more lighting |
| | markers and/or other markers such as audible markers |
| | and proximity sensors and wireless transmitters. |
| 141 | RFID-antenna |
| 142 | Sensor mounting bracket including 2 range finder sensors |
| 150 | A patient room with several beds 151a-151d |
| 152 | A sensor and light source. The light source can be a marker |
| | light. The sensor can be any kind of proximity sensor. In one |
| | example it is a camera. |
| 152a | A first sensor and light source. The light source can be a |
| | marker light. The sensor can be any kind of proximity |
| | sensor. In one example it is a camera. |
| 152b | A second sensor and light source. The light source can be a |
| | marker light. The sensor can be any kind of proximity sensor. |
| | In one example it is a camera. |
| 152c | A third sensor and light source. The light source can be a |
| | marker light. The sensor can be any kind of proximity |
| | sensor. In one example it is a camera. |
| 152d | A fourth sensor and light source. The light source can be a |
| | marker light. The sensor can be any kind of proximity sensor. |
| | In one example it is a camera. |
| 153a | Sensor zone A associated with first bed. |
| 153b | Sensor zone B associated with second bed. |
| 153c | Sensor zone C associated with third bed. |
| 153d | Sensor zone D associated with fourth bed. |
| 154 | Red light marker, i.e., person associated with a red marker has |
| | not followed the requested regime for hand wash hygiene. |
| 154a | Red light marker, i.e., person associated with the red marker in |
| | zone A has not followed the requested regime for hand wash |
| | hygiene. |
| 154d | Red light marker, i.e., person associated with the red marker in |
| | zone D has not followed the requested regime for hand wash |
| | hygiene. |
| 154e | Red light marker, i.e., person associated with the red marker in |
| | zone E-entrance area-has not followed the requested regime |
| | for hand wash hygiene. |
| 155 | Green light marker, i.e., person associated with a green marker |
| | has followed the requested regime for hand wash hygiene. |
| 155a | Green light marker, i.e., person associated with the green |
| | marker in zone A has followed the requested regime for hand |
| | wash hygiene. |
| 155b | Green light marker, i.e., person associated with the green |
| | marker in zone A has followed the requested regime for hand |
| | wash hygiene. |
| 155c | Green light marker, i.e., person associated with the green |
| | marker in zone A has followed the requested regime for hand |
| | wash hygiene. |
| 156a | A first bed. |
| 156b | A second bed |
| 156c | A third bed |
| 156d | A fourth bed |
| 157a | A path of a first person ‘a’ from entrance to the first bed 156a |
| 157b | A path of a second person ‘b’ from entrance to the second bed |
| | 156b. |
| 157c | A path of a third person ‘c’ from first bed 156a to the entrance |
| | area. |
| 157d | A path of a fourth person ‘d’ from entrance to the third bed |
| | 156c and further to the fourth bed. |
| 158 | A first sink |
| 159 | A second sink |
| 161 | Sensor and associated processor including wireless |
| | communication unit. |
| 170 | Light source with a split light characteristic, designed to flash |
| | with different colours to a left-and right-hand side of a bed. |
| 200 | Patient room with smart monitoring using a sensor in |
| | communication with a processor. |
| 201 | A sensor mounted in the ceiling of a patient room. |
| 202 | A zone in the patient room monitoring a washing station |
| | including one or more automatic hand disinfection dispensers |
| | and a washing sink. |
| 203 | A zone in the patient room covering the area in front of the |
| | entrance to a bathroom. |
| 204 | A distance meter, i.e. a sensor that detects the distance of a |
| | person from the sensor. |
| 205 | A zone in the patient room covering the area in front of the |
| | entrance to the patient room 200. |
| a | First person |
| b | Second person |
| c | Third person |
| d | Fourth person |
| w₁ | Overlap between sensor zone 153a and sensor zone 153d. |
| w₂ | Overlap between sensor zone 153b and sensor zone 153c. |
| z₁ | Overlap between sensor zone 153a and sensor zone 153b. |
| z₂ | Overlap between sensor zone 153c and sensor zone 153d. |

1. Automatic hand disinfection dispenser at least comprising:
a. a housing that can be opened and closed, where the housing at least comprises:
i. two spray chambers each with an open free end adapted to receive a hand and each with a closed end, the two spray chambers further comprise several spray nozzles;
ii. a removable disinfectant container;
iii. a power source providing electric power for operation of the automatic hand disinfection dispenser;
iv. a control unit;
v. “hand sensors”;
vi. a fluidic system, and
wherein the two hand sensors are configured to detect hands being inserted into the two spray chambers, If hand sensors are active “1” then the hand sensor is configured to transmit a trigger signal to the control unit of the automatic hand disinfection dispenser, the control unit is configured to initiate dispensing from the several spray nozzles of the two spray chambers in response to the received trigger signal, where the fluidic system is configured to transport disinfectant from the removable disinfectant container to the several spray nozzles, and the fluidic system is also configured to generate pressure to spray the disinfectant onto a user's hands being inserted into the two spray chambers.

2. Automatic hand disinfection dispenser according to claim 1, where the housing further comprises at least:
vii. a proximity sensor for detection of personnel in proximity of the automatic hand disinfection dispenser.

3. Automatic hand disinfection dispenser according to claim 2 where the communication unit is one of:
a Wi-Fi unit in compliance with Wi-Fi standards;
cellular network in compliance with one of: GSM, GPRS, EDGE, WCDMA, 3G, 4G, 5G and H+;
Bluetooth;
Zigbee;
BLE;
RFID;
Z wave; and
LPWANS.

4. Automatic hand disinfection dispenser according to claim 1, where the housing further comprises one or more of:
viii. at least one lightening source and user interface, where the lightening source and the user interface at least comprises:
1. a group of lightening indicators visible for a user which is faced towards the front end of the automatic hand disinfection dispenser and not more than two meters away from the two spray chambers;
2. a beacon light displaying status information of the automatic hand disinfection dispenser, and
3. an internal interface, where the internal interface provides necessary controls and signals for maintenance purpose of the automatic hand disinfection dispenser;
ix. at least one RFID reader;
X. one or more audible sources;
xi. a metal detector integrated into each of the two spray chambers;
xii. a disinfectant level sensor, detecting whether the disinfectant level falls below a certain threshold in the removable disinfectant container;
xiii. a disinfectant label scanner, the scanner scans a fixed area of the removable disinfectant container, the scanner is configured to scan 2D-codes, if a 2D code is present its data is saved in a memory of the automatic hand disinfection dispenser and wirelessly transmitted to an external access point;
xiv. a removable dripping tray;
xv. one or more memory circuits in communication with the control unit;
xvi. a housing open sensor, detects opening of the substantially closed housing;
xvii. a housing lock for unlocking and locking the substantially closed housing;
xviii. a mode switch button, and
xix. an ambient light sensor.

5. Automatic hand disinfection dispenser according to claim 1, wherein the automatic hand disinfection dispenser is configurable through a Wi-Fi interface from an external access point.

6. Automatic hand disinfection dispenser according to claim 4, where the automatic hand disinfection dispenser is configurable from an external access point and configuration of the automatic hand disinfection dispenser includes one or more of the configuration parameters:
a. date and time;
b. user identification via RFID selectable to: on or off;
c. metal detection selectable to: on or off;
d. night mode, selectable start time for night mode and selectable stop time for night mode;
e. selectable audio volume;
f. all audible feedback is selectable to: on or off;
g. audible feedback user identification is selectable to: on or off;
h. negative audible feedback after metal detection is selectable to: on or off;
i. time delay from hand detection to start of spraying is selectable;
j. time delay when metal is detected for one or two hands from hand detection to start of spraying is selectable;
k. audible feedback after successful dispensing is selectable to one of: off and confirmation tone and audio file
l. time delay from end of dispensing to the restart of the hand disinfection process—if proximity sensor does not register that the user has left the automatic hand disinfection dispenser—is selectable;
m. audible feedback upon entering or exiting cleaning mode is selectable; and
n. number of cycles sprayed by the one removable disinfectant container until a level warning is activated is selectable.

7. Automatic hand disinfection dispenser according to claim 4, where the automatic hand disinfection dispenser is configured to enter cleaning mode by activation of a push button on the housing.

8. A method for operation of an automatic hand disinfection dispenser including two spray chambers including the steps of:
a. a first user approaches the automatic hand disinfection dispenser and the automatic hand disinfection dispenser detects the approach utilising an embedded proximity sensor;
b. the automatic hand disinfection dispenser initiates an insert hand signal indicating that the first user shall insert hands into the two spray chambers;
c. in each spray chamber detecting by a hand sensor a hand being inserted into the spray chamber and the hand sensors triggers the automatic hand disinfection dispenser to enter spraying modes when hands are detected in one or both spray chambers;
d. the first user removes his disinfected hands and moves away when spraying process is finished.

9. A method according to claim 8, wherein step b further comprises the steps of: switching on a hand insertion LED light on the front of the automatic hand disinfection dispenser statically and starting to flash or run upwards a light strip.

10. A method according to claim 8, wherein step c further comprises the sub steps of: the automatic hand disinfection dispenser signals that it is in spraying mode by switching on a light strip statically, by turning off a hand insertion icon.

11. Distributed hand hygiene assistance system at least comprising the following units:
a. at least one access point configured to receive and transmit data from several automatic hand disinfection dispensers, the access point further being configured to send commands to one or more automatic hand disinfection dispensers;
b. at least one or more markers warning personnel about violation of hygiene rules; and
c. at least one automatic hand disinfection dispenser where the automatic hand disinfection dispenser at least comprises:
i. a housing that can be opened and closed and where the housing at least comprises:
1. two spray chambers each with an open free end adapted to receive a hand and each with a closed end, the two spray chambers further comprises several spray nozzles;
2. a disinfectant container;
3. a power source providing electric power for operation of the automatic hand disinfection dispenser, and
4. a communication unit for wireless communication.

12. The distributed hand hygiene assistance system according to claim 11, where the system further comprises one or more of the following units:
c. at least one motion detector for the detection of persons, the motion detector is fixedly mounted,
d. at least one marker lights being visible and indicating whether a set hygiene regime has been followed or not;
e. at least one wireless communication device associated with the at least one motion detector and the at least one marker lights source;
and where the at least one marker is marker lights at least one marker lights being visible and indicating any violation of hygiene rules and where the automatic hand disinfection dispenser is at least configured to communicate with the motion detector.

13. The distributed hand hygiene assistance system according to claim 12, where the at least one motion detector is one or more of: a video camera, an IR-sensor, a PIR-sensor, laser motion sensor, microwave sensor and a lidar.

14. The distributed hand hygiene assistance system according to claim 12, where:
a. a detection housing includes one motion detector, one processor, one wireless communication device, and at least one marker lights source,
b. the one wireless communication device is at least configured to communicate with the automatic hand disinfection dispenser,
c. the motion detector is connected with the processor for direct communication with the processor,
d. the processor is connected with the at least one marker lights, and
the processor is configured to send commands to the at least one marker light, the commands are at least:
if hygiene regime is violated send a trigger signal to the at least one marker lights to switch on an alerting light indicating the violation of the hygiene regime,
if hygiene regime is followed send a trigger signal to the at least one marker lights to switch on a light indicating that the hygiene regime is followed.

15. The distributed hand hygiene assistance system according to claim 11, where the system further at least comprises an RFID reader for registration of users associated with unique RFID chips.
